(19) United States
(12) Patent Application Publication
(10) Pub. No.: US 2006/0173046 A1
(43) Pub. Date: Aug. 3, 2006

(54) Hydroxypyridine cgrp receptor antagonists

(76) Inventors: Ian M. Bell, Harleysville, PA (US); Steven N. Gallicchio, Horsham, PA (US); C Blair Zartman, Hatfield, PA (US)

Correspondence Address: MERCK AND CO., INC, P O BOX 2000, RAHWAY, NJ, 07065-0907, US

(21) Appl. No.: 10/564,706

(22) PCT Filed: Jul. 9, 2004
(86) PCT No.: PCT/US04/21888

§ 371 Date: Jan. 13, 2006

Related U.S. Application Data
(60) Provisional application No. 60/487,316, filed on Jul. 15, 2003.

(51) Int. Cl. A61K 31/4439; A61K 31/4415; C07D 403/02

(52) U.S. Cl. 514/340; 514/346; 546/291; 546/268.4

(57) ABSTRACT

The present invention is directed to compounds of Formula I: I (where variables R¹, R², R³ and R⁴ are as defined herein) useful as antagonists of CGRP receptors and useful in the treatment or prevention of diseases in which CGRP is involved, such as headache, migraine and cluster headache. The invention is also directed to the use of such compounds as ligands of AM receptors for the treatment or prevention of diseases in which AM is involved, such as cancer. The invention is further directed to pharmaceutical compositions comprising these compounds and the use of these compounds and compositions in the prevention or treatment of such diseases in which CGRP and/or AM are involved.

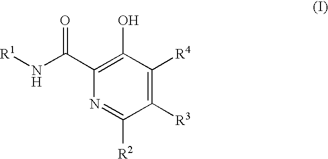

### BACKGROUND OF THE INVENTION

CGRP (Calcitonin Gene-Related Peptide) is a naturally occurring 37-amino acid peptide that is generated by tissue-specific alternate processing of calcitonin messenger RNA and is widely distributed in the central and peripheral nervous system. CGRP is localized predominantly in sensory afferent and central neurons and mediates several biological actions, including vasodilation. CGRP is expressed in alpha- and beta-forms that vary by one and three amino acids in the rat and human, respectively. CGRP-alpha and CGRP-beta display similar biological properties. When released from the cell, CGRP initiates its biological responses by binding to specific cell surface receptors that are predominantly coupled to the activation of adenylyl cyclase. CGRP receptors have been identified and pharmacologically evaluated in several tissues and cells, including those of brain, cardiovascular, endothelial, and smooth muscle origin.

CGRP is a potent vasodilator that has been implicated in the pathology of cerebrovascular disorders such as migraine and cluster headache. In clinical studies, elevated levels of CGRP in the jugular vein were found to occur during migraine attacks (Goadsby et al., Ann. Neurol., 1990, 28, 183-187). CGRP activates receptors on the smooth muscle of intracranial vessels, leading to increased vasodilation, which is thought to be the major source of headache pain during migraine attacks (Lance, Headache Pathogenesis: Monoamines, Neuropeptides, Purines and Nitric Oxide, Lippincott-Raven Publishers, 1997, 3-9). The middle meningeal artery, the principle artery in the dura mater, is innervated by sensory fibers from the trigeminal ganglion which contain several neuropeptides, including CGRP. Trigeminal ganglion stimulation in the cat resulted in increased levels of CGRP, and in humans, activation of the trigeminal system caused facial flushing and increased levels of CGRP in the external jugular vein (Goadsby et al., Ann. Neurol., 1988, 23, 193-196). Electrical stimulation of the dura mater in rats increased the diameter of the middle meningeal artery, an effect that was blocked by prior administration of CGRP(8-37), a peptide CGRP antagonist (Williamson et al., Cephalalgia, 1997, 17, 525-531). Trigeminal ganglion stimulation increased facial blood flow in the rat, which was inhibited by CGRP(8-37) (Escott et al., Brain Res. 1995, 669, 93-99). Electrical stimulation of the trigeminal ganglion in marmoset produced an increase in facial blood flow that could be blocked by the non-peptide CGRP antagonist BIBN4096BS (Doods et al., Br. J. Pharmacol., 2000, 129, 420423). Thus the vascular effects of CGRP may be attenuated, prevented or reversed by a CGRP antagonist.

CGRP-mediated vasodilation of rat middle meningeal artery was shown to sensitize neurons of the trigeminal nucleus caudalis (Williamson et al., The CGRP Family: Calcitonin Gene-Related Peptide (CGRP), Amylin, and Adrenomedullin, Landes Bioscience, 2000, 245-247). Similarly, distention of dural blood vessels during migraine headache may sensitize trigeminal neurons. Some of the associated symptoms of migraine, including extra-cranial pain and facial allodynia, may be the result of sensitized trigeminal neurons (Burstein et al., Ann. Neurol. 2000, 47, 614-624). A CGRP antagonist may be beneficial in attenuating, preventing or reversing the effects of neuronal sensitization.

The ability of the compounds of the present invention to act as CGRP antagonists makes them useful pharmacological agents for disorders that involve CGRP in humans and animals, but particularly in humans. Such disorders include migraine and cluster headache (Doods, Curr Opin Inves Drugs, 2001, 2 (9), 1261-1268; Edvinsson et al., Cephalalgia, 1994, 14, 320-327); chronic tension type headache (Ashina et al., Neurology, 2000, 14, 1335-1340); pain (Yu et al., Eur. J. Pharm., 1998, 347, 275-282); chronic pain (Hulsebosch et al., Pain, 2000, 86, 163-175); neurogenic inflammation and inflammatory pain (Holzer, Neurosci., 1988, 24, 739-768; Delay-Goyet et al., Acta Physiol. Scanda. 1992, 146, 537-538; Salmon et al., Nature Neurosci., 2001, 4(4), 357-358); eye pain May et al. Cephalalgia, 2002, 22, 195-196), tooth pain (Awawdeh et al., Int. Endocrin. J., 2002, 35, 30-36), non-insulin dependent diabetes mellitus (Molina et al., Diabetes, 1990, 39, 260-265); vascular disorders; inflammation (Zhang et al., Pain, 2001, 89, 265), arthritis, bronchial hyperreactivity, asthma, Foster et al., Ann. NY Acad. Sci., 1992, 657, 397-404; Schini et al., Am. J. Physiol., 1994, 267, H2483-H2490; Zheng et al., J. Virol., 1993, 67, 5786-5791); shock, sepsis (Beer et al., Crit. Care Med., 2002, 30 (8), 1794-1798); opiate withdrawal syndrome (Salmon et al., Nature Neurosci., 2001, 4(4), 357-358) morphine tolerance (Menard et al., J. Neurosci., 1996, 16 (7), 2342-2351); hot flashes in men and women (Chen et al., Lancet, 1993, 342, 49; Spetz et al., J. Urology, 2001, 166, 1720-1723); allergic dermatitis (Wallengren, Contact Dermatitis, 2000, 43 (3), 137-143); psoriasis; encephalitis, brain trauma, ischaemia, stroke, epilepsy, and neurodegenerative diseases (Rohrenbeck et al., Neurobiol. of Disease 1999, 6, 15-34); skin diseases (Geppetti and Holzer, Eds., Neurogenic Inflammation, 1996, CRC Press, Boca Raton, Fla.), neurogenic cutaneous redness, skin rosaceousness and erythema; tinnitus (Herzog et al., J. Membrane Biology, 2002, 189(3), 225); inflammatory bowel disease, irritable bowel syndrome, (Hoffman et al. Scandinavian Journal of Gastroenterology, 2002, 37(4) 414422) and cystitis. Of particular importance is the acute or prophylactic treatment of headache, including migraine and cluster headache.

The present invention relates to compounds that are useful as ligands for CGRP receptors, in particular antagonists for CGRP receptors, processes for their preparation, their use in therapy, pharmaceutical compositions comprising them and methods of therapy using them.

### SUMMARY OF THE INVENTION

The present invention is directed to compounds of Formula I: 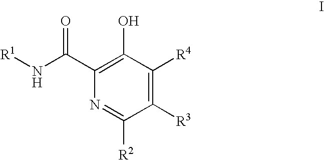
(where variables R¹, R², R³ and R⁴ are as defined herein) useful as antagonists of CGRP receptors and useful in the treatment or prevention of diseases in which CGRP is involved, such as headache, migraine and cluster headache. The invention is also directed to the use of such compounds as ligands of AM receptors for the treatment or prevention of diseases in which AM is involved, such as cancer. The invention is further directed to pharmaceutical compositions comprising these compounds and the use of these compounds and compositions in the prevention or treatment of such diseases in which CGRP and/or AM are involved.

### DETAILED DESCRIPTION OF THE INVENTION

The present invention is directed to compounds of Formula I: 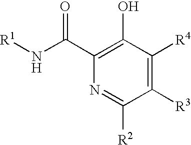
wherein:
R¹ is selected from:
   a) hydrogen,
   b) aryl, heterocycle, C₃-C₁₀ cycloalkyl, C₂-C₆ alkenyl, C₂-C₆ alkynyl, and
   c) C₁-C₆ alkyl, unsubstituted or substituted with 1 to 5 substituents selected from:
      1) aryl, unsubstituted or substituted with 1 to 5 substituents selected from:
         i) C₁-C₆ alkyl, unsubstituted or substituted with 1-3 fluoro,
         ii) C₃-C₆ cycloalkyl,
         iii) C₂-C₆ alkynyl,
         iv) OR¹⁰,
         v) aryl,
         vi) heterocycle,
         vii) CN, and
         viii) halo;
      2) heterocycle, unsubstituted or substituted with 1 to 5 substituents selected from:
         i) C₁-C₆ alkyl, unsubstituted or substituted with 1-3 fluoro,
         ii) —OR¹⁰,
         iii) aryl, and
         iv) halo;
      3) C₃-C₁₀ cycloalkyl,
      4) C₂-C₆ alkenyl,
      5) C₂-C₆ alkynyl,
      6) —OR¹⁰,
      7) —S(O)ₘR¹¹,
      8) —NR⁶—C(O)R⁷,
      9) —C(O)—N(R⁶)(R⁷),
      10) —CN,
      11) —NR⁶—C(O)—N(R⁶)(R⁷),
      12) —C(O)—OR¹⁰,
      13) halo, and
      14) —N(R⁶)(R⁷);
R² is selected from:
   a) —NR⁶—C(O)R⁷,
   b) —NR⁶—S(O)₂R⁷, and
   c) —NR⁶—S(O)²—N⁶)(R⁷);
R³ and R⁴ are independently selected from:
hydrogen, aryl, heterocycle, halo, C₁-C₆ alkyl, C₃-C₁₀ cycloalkyl, C₂-C₆ alkenyl, C₂-C₆ alkynyl, C₁-C₄ haloalkyl, R¹⁰O—, R¹¹S(O)ₘ—, R⁶C(O)—NR⁷—, CN, (R⁶)(R⁷)N—C(O)—(NR⁶)—, (R⁶)(R⁷)—N—C(O)—, R¹⁰C(O)—,R¹⁰OC(O)—, and N(R⁶)(R⁷); or
wherein R³ and R⁴ are optionally joined to form a saturated or unsaturated ring, containing 0-3 heteroatoms, wherein said ring is phenyl, pyridyl, pyrimidinyl, pyrazinyl, thiophenyl, furanyl, imidazolyl, thiazolyl, oxazolyl, and triazolyl, as well as partially saturated analogues thereof, said ring optionally substituted with one or more of:
   aryl, heterocycle, C₁-C₆ alkyl, C₃-C₁₀ cycloalkyl, C₂-C₆ alkynyl, R¹⁰O—, R¹¹S(O)ₘ—, R⁶C(O)NR⁷—, R⁶S(O)₂NR⁷—, (R⁶)(R⁷)N—C(O)—, CN, R¹⁰OC(O)—, F, and —N(R⁶)(R⁷);
R⁶ and R⁷ are independently selected from hydrogen, C₁-C₆ alkyl, C₃-C₁₀ cycloalkyl, heterocycle, aryl, unsubstituted or substituted with one or more of:
   a) C₁-C₄ alkyl,
   b) C₁-C₄ alkoxy,
   c) aryl or heterocycle,
   d) halo,
   e) —OR¹⁰, and
   f) —N(R¹⁰)₂;
wherein R⁶ and R⁷ may be joined to form a ring;
R¹⁰ is independently selected from hydrogen, C₁-C₆ alkyl, —CF₃, C₃-C₁₀ cycloalkyl, benzyl, and aryl;
R¹¹ is independently selected from C₁-C₆ alkyl, and aryl;
m is 0, 1, or 2;
and pharmaceutically acceptable salts and individual diastereomers thereof.

In an embodiment of the present invention, R¹ is —CH₂-aryl, unsubstituted or substituted with 1-3 substituents selected from: fluoro, chloro, bromo, iodo and methyl.

In an embodiment of the present invention, R¹ is benzyl, substituted with 1-3 fluoro.

In an embodiment of the present invention, R¹ is —CH₂C(O)OR¹⁰.

In an embodiment of the present invention, R¹ is —CH₂C(O)OC(CH₃)₃.

In an embodiment of the present invention, R¹ is —CH₂C(O)NHR⁶.

In an embodiment of the present invention, R¹ is —CH₂C(O)NH(C₄-C₁₀ cycloalkyl).

In an embodiment of the present invention, R¹ is —CH₂C(O)NH-aryl.

In an embodiment of the present invention, R² is —NR⁶S(O)₂R⁷.

In an embodiment of the present invention, R³ is hydrogen.

In an embodiment of the present invention, R³ and R⁴ are joined to form a ring selected from: phenyl, pyridyl, pyrimidinyl and pyrazinyl.

In an embodiment of the present invention, R³ and R⁴ are joined to form a pyridyl ring.

In an embodiment of the present invention, R⁴ is bromo.

In an embodiment of the present invention, R⁴ is —C(O)OR¹⁰.

It is to be understood that where one or more of the above recited structures or substructures recite multiple substituents having the same designation each such variable may be the same or different from each similarly designated variable. For example, R² is recited four times in formula I, and each R² in formula I may independently be any of the substructures defined under R². The invention is not limited to structures and substructures wherein each R² must be the same for a given structure. The same is true with respect to any variable appearing multiple time in a structure or substructure.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds.

Some of the compounds described herein contain olefinic double bonds, and unless specified otherwise, are meant to include both E and Z geometric isomers.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As will be appreciated by those of skill in the art, not all of the R¹⁰ and R¹¹ substituents are capable of forming a ring structure. Moreover, even those substituents capable of ring formation may or may not form a ring structure.

Also as appreciated by those of skill in the art, halo or halogen as used herein are intended to include chloro, fluoro, bromo and iodo.

As used herein, “alkyl” is intended to mean linear, branched and cyclic structures having no double or triple bonds. Thus C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl and hexyl. “Cycloalkyl” is an alkyl, part or all of which which forms a ring of three or more atoms. C₀ or C₀alkyl is defined to identify the presence of a direct covalent bond.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. Thus C₂₋₆alkynyl is defined to identify the group as having 2, 3, 4, 5 or 6 carbons in a linear or branched arrangement, such that C₂₋₆alkynyl specifically includes 2-hexynyl and 2-pentynyl.

As used herein, “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl.

The term “heterocycle” or “heterocyclic”, as used herein except where noted, represents a stable 5- to 7-membered monocyclic- or stable 8- to 11-membered bicyclic heterocyclic ring system which is either saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heterocyclic groups include, but are not limited to, azetidine, chroman, dihydrofuran, dihydropyran, dioxane, dioxolane, hexahydroazepine, imidazolidine, imidazolidinone, imidazoline, imidazolinone, indoline, isochroman, isoindoline, isothiazoline, isothiazolidine, isoxazoline, isoxazolidine, morpholine, morpholinone, oxazoline, oxazolidine, oxazolidinone, oxetane, 2-oxohexahydroazepin, 2-oxopiperazine, 2-oxopiperidine, 2-oxopyrrolidine, piperazine, piperidine, pyran, pyrazolidine, pyrazoline, pyrrolidine, pyrroline, quinuclidine, tetrahydrofuran, tetrahydropyran, thiamorpholine, thiazoline, thiazolidine, thiomorpholine and N-oxides thereof.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

The term “alkoxy,” as in C₁-C₆ alkoxy, is intended to refer to include alkoxy groups of from 1 to 6 carbon atoms of a straight, branched and cyclic configuration. Examples include methoxy, ethoxy, propoxy, isopropoxy, cyclopropyloxy, cyclohexyloxy and the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The number of certain variables present in certain instances is defined in terms of the number of carbons present. For example, variable “p” is occasionally defined as follows: “p is 0 to 2q+1, for a substituent with q carbons”. Where the substituent is “(F)ₚC₁₋₃ alkyl” this means that when there is one carbon, there are 2(1)+1=3 fluorines. When there are two carbons, there are 2(2)+1=5 fluorines, and when thre are three carbons there are 2(3)=1=7 fluorines.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the invention the salts are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of antagonism of CGRP receptors in a patient such as a mammal in need of such antagonism comprising the administration of an effective amount of the compound. The present invention is directed to the use of the compounds disclosed herein as antagonists of CGRP receptors. In addition to primates, especially humans, a variety of other mammals can be treated according to the method of the present invention.

Another embodiment of the present invention is directed to a method for the treatment, control, amelioration, or reduction of risk of a disease or disorder in which the CGRP receptor is involved in a patient that comprises administering to the patient a therapeutically effective amount of a compound that is an antagonist of CGRP receptors.

The present invention is further directed to a method for the manufacture of a medicament for antagonism of CGRP receptors activity in humans and animals comprising combining a compound of the present invention with a pharmaceutical carrier or diluent.

The subject treated in the present methods is generally a mammal, for example a human being, male or female, in whom antagonism of CGRP receptor activity is desired. The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as antagonists of CGRP receptor activity may be demonstrated by methodology known in the art. Inhibition of the binding of ¹²⁵I-CGRP to receptors and functional antagonism of CGRP receptors were determined as follows:

NATIVE RECEPTOR BINDING ASSAY: The binding of ¹²⁵I-CGRP to receptors in SK-N-MC cell membranes was carried out essentially as described (Edvinsson et al. (2001) Eur. J. Pharmacol. 415, 39-44). Briefly, membranes (25 μg) were incubated in 1 ml of binding buffer [10 mM HEPES, pH 7.4,5 MM MgCl₂ and 0.2% bovine serum albumin (BSA)] containing 10 pM ¹²⁵I-CGRP andantagonist. After incubation at room temperature for 3 h, the assay was terminated by filtration through GFB glass fibre filter plates (Millipore) that had been blocked with 0.5% polyethyleneimine for 3 h. The filters were washed three times with ice-cold assay buffer, then the plates were air dried. Scintillation fluid (50 μl) was added and the radioactivity was counted on a Topcount (Packard Instrument). Data analysis was carried out by using Prism and the Kᵢ was determined by using the Cheng-Prusoff equation (Cheng & Prusoff (1973) Biochem Pharmacol. 22,3099-3108).

NATIVE RECEPTOR FUNCTIONAL ASSAY: SK-N-MC cells were grown in minimal essential medium (MEM) supplemented with 10% fetal bovine serum, 2 mM L-glutamine, 0.1 mM non-essential amino acids, 1 mM sodium pyruvate, 100 units/ml penicillin and 100 μg/ml streptomycin at 37° C., 95% humidity, and 5% CO₂. For cAMP assays, cells were plated at 5×10⁵ cells/well in 96-well poly-D-lysine-coated plates (Becton-Dickinson) and cultured for ˜18 h before assay. Cells were washed with phosphate-buffered saline (PBS, Sigma) then pre-incubated with 300 μM isobutylmethylxanthine in serum-free MEM for 30 min at 37° C. Antagonist was added and the cells were incubated for 10 min before the addition of CGRP. The incubation was continued for another 15 min, then the cells were washed with PBS and processed for cAMP determination according to the manufacturer's recommended protocol. Maximal stimulation over basal was defined by using 100 nM CGRP. Dose-response curves were generated by using Prism. Dose-ratios (DR) were calculated and used to construct full Schild plots (Arunlakshana & Schild (1959) Br. J. Pharmacol. 14,48-58).

RECOMBINANT RECEPTOR: Human CRLR (Genbank accession number L76380) was subcloned into the expression vector pIREShyg2 (BD Biosciences Clontech) as a 5′NheI and 3′PmeI fragment. Human RAMP1 (Genbank accession number AJ001014) was subcloned into the expression vector pIRESpuro2 (BD Biosciences Clontech) as a 5′NheI and 3′NotI fragment. 293 cells (human embryonic kidney cells; ATCC #CRL-1573) were cultured in DMEM with 4.5 g/L glucose, 1 mM sodium pyruvate and 2 mM glutamine supplemented with 10% fetal bovine serum (FBS), 100 units/nL penicillin and 100 ug/ml streptomycin, and maintained at 37° C. and 95% humidity. Cells were subcultured by treatment with 0.25% trypsin with 0.1% EDTA in HBSS. Stable cell line generation was accomplished by co-transfecting 10 ug of DNA with 30 ug Lipofectamine 2000 (Invitrogen) in 75 cm² flasks. CRLR and RAMP1 expression constructs were co-transfected in equal amounts. Twenty-four hours after transfection the cells were diluted and selective medium (growth medium +300 ug/ml hygromycin and 1 ug/ml puromycin) was added the following day. A clonal cell line was generated by single cell deposition utilizing a FACS Vantage SE (Becton Dickinson). Growth medium was adjusted to 150 ug/ml hygromycin and 0.5 ug/ml puromycin for cell propagation.

RECOMBINANT RECEPTOR BINDING ASSAY: Cells expressing recombinant human CRLR/RAMP1 were washed with PBS and harvested in harvest buffer containing 50 mM HEPES, 1 mM EDTA and Complete protease inhibitors (Roche). The cell suspension was disrupted with a laboratory homogenizer and centrifuged at 48,000 g to isolate membranes. The pellets were resuspended in harvest buffer plus 250 mM sucrose and stored at −70° C. For binding assays, 10 ug of membranes were incubated in 1 ml binding buffer (10 mM HEPES, pH 7.4, 5 mM MgCl₂, and 0.2% BSA) for 3 hours at room temperature containing 10 pM ¹²⁵I-hCGRP (Amersham Biosciences) and antagonist. The assay was terminated by filtration through 96-well GFB glass fiber filter plates (Millipore) that had been blocked with 0.05% polyethyleneimine. The filters were washed 3 times with ice-cold assay buffer (10 mM HEPES, pH 7.4). Scintillation fluid was added and the plates were counted on a Topcount (Packard). Non-specific binding was determined and the data analysis was carried out with the apparent dissociation constant (Kᵢ) determined by using a non-linear least squares fitting the bound CPM data to the equation below:

$${Y}_{obsd} = \frac{\begin{matrix}( {Y}_{ max} - {Y}_{ min} ) ( \% {I}_{ max} - \% {I}_{ min} / 100 ) + {Y}_{ min} + \\ ( {Y}_{ max} - {Y}_{ min} ) ( 100 - \% {I}_{ max} / 100 )\end{matrix}}{1 + ( [ Drug ] / {{K}_{i} ( 1 + [ Radiolabel ] / {K}_{d} )}^{nH}}$$

Where Y is observed CPM bound, Yₘₐₓ is total bound counts, Y min is non specific bound counts, (Y max−Y min) is specific bound counts, % I max is the maximum percent inhibition, % I min is the minimum percent inhibition, radiolabel is the probe, and the K_{d} is the apparent dissociation constant for the radioligand for the receptor as determined by Hot saturation experiments.

RECOMBINANT RECEPTOR FUNCTIONAL ASSAY: Cells were plated in complete growth medium at 85,000 cells/well in 96-well poly-D-lysine coated plates (Corning) and cultured for ˜19 h before assay. Cells were washed with PBS and then incubated with inhibitor for 30 min at 37° C. and 95% humidity in Cellgro Complete Serum-Free/Low-Protein medium (Mediatech, Inc.) with L-glutamine and 1 g/L BSA. Isobutyl-methylxanthine was added to the cells at a concentration of 300 μM and incubated for 30 min at 37° C. Human α-CGRP was added to the cells at a concentration of 0.3 nM and allowed to incubate at 37° C. for 5 min. After a-CGRP stimulation the cells were washed with PBS and processed for cAMP determination utilizing the two-stage assay procedure according to the manufacturer's recommended protocol (cAMP SPA direct screening assay system; RPA 559; Amersham Biosciences). Dose response curves were plotted and IC₅₀ values determined from a 4-parameter logistic fit as defined by the equation y=((a−d)/(1+(x/c)^{b})+d, where y=response, x=dose, a=max response, d=min response, c=inflection point and b=slope.

In particular, the compounds of the following examples had activity as antagonists of the CGRP receptor in the aforementioned assays, generally with a Kᵢ or IC₅₀ value of less than about 50 μM. Such a result is indicative of the intrinsic activity of the compounds in use as antagonists of CGRP receptors.

The ability of the compounds of the present invention to act as CORP antagonists makes them useful pharmacological agents for disorders that involve CGRP in humans and animals, but particularly in humans.

The compounds of the present invention have utility in treating, preventing, ameliorating, controlling or reducing the risk of one or more of the following conditions or diseases: headache; migraine; cluster headache; chronic tension type headache; pain; chronic pain; neurogenic inflammation and inflammatory pain; neuropathic pain; eye pain; tooth pain; diabetes; non-insulin dependent diabetes mellitus; vascular disorders; inflammation; arthritis; bronchial hyperreactivity, asthma; shock; sepsis; opiate withdrawal syndrome; morphine tolerance; hot flashes in men and women; allergic dermatitis; psoriasis; encephalitis; brain trauma; epilepsy; neurodegenerative diseases; skin diseases; neurogenic cutaneous redness, skin rosaceousness and erythema; inflammatory bowel disease, irritable bowel syndrome, cystitis; and other conditions that may be treated or prevented by antagonism of CGRP receptors. Of particular importance is the acute or prophylactic treatment of headache, including migraine and cluster headache.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

For example, the present compounds may be used in conjunction with an anti-inflammatory or analgesic agent or an anti-migraine agent, such as an ergotamine or 5-HT₁ agonists, especially a 5-HT_{1B/1D} agonist, for example sumatriptan, naratriptan, zolmitriptan, eletriptan, almotriptan, frovatriptan, donitriptan, and rizatriptan; a cyclooxygenase inhibitor, such as a selective cyclooxygenase-2 inhibitor, for example rofecoxib, etoricoxib, celecoxib, valdecoxib or paracoxib; a non-steroidal anti-inflammatory agent or a cytokine-suppressing anti-inflammatory agent, for example with a compound such as aspirin, ibuprofen, ketoprofen, fenoprofen, naproxen, indomethacin, sulindac, meloxicam, piroxicam, tenoxicam, lornoxicam, ketorolac, etodolac, mefenamic acid, meclofenamic acid, flufenamic acid, tolfenamic acid, diclofenac, oxaprozin, apazone, nimesulide, nabumetone, tenidap, etanercept, tolmetin, phenylbutazone, oxyphenbutazone, diflunisal, salsalate, olsalazine or sulfasalazine and the like; or a steroidal analgesic. Similarly, the instant compounds may be administered with a pain reliever such as acetaminophen, phenacetin, codeine, fentanyl, sufentanil, methadone, acetyl methadol, buprenorphine or morphine.

Additionally, the present compounds may be used in conjunction with an interleukin inhibitor, such as an interleukin-1 inhibitor; an NK-1 receptor antagonist, for example aprepitant; an NMDA antagonist; an NR2B antagonist; a bradykinin-1 receptor antagonist; an adenosine A1 receptor agonist; a sodium channel blocker, for example lamotrigine; an opiate agonist such as levomethadyl acetate or methadyl acetate; a lipoxygenase inhibitor, such as an inhibitor of 5-lipoxygenase; an alpha receptor antagonist, for example indoramin; an alpha receptor agonist; a vanilloid receptor antagonist; an mGluR5 agonist, antagonist or potentiator; a GABA A receptor modulator, for example acamprosate calcium; nicotinic antagonists or agonists including nicotine; muscarinic agonists or antagonists; a selective serotonin reuptake inhibitor, for example fluoxetine, paroxetine, sertraline, duloxetine, escitalopram, or citalopram; a tricyclic antidepressant, for example amitriptyline, doxepin, protriptyline, desipramine, trimipramine, or imipramine; a leukotriene antagonist, for example montelukast or zafirlukast; an inhibitor of nitric oxide or an inhibitor of the synthesis of nitric oxide.

Also, the present compounds may be used in conjunction with ergot alkaloids, for example ergotamine, ergonovine, ergonovine, methylergonovine, metergoline, ergoloid mesylates, dihydroergotamine, dihydroergocornine, dihydroergocristine, dihydroergocryptine, dihydro-α-ergocryptine, dihydro-β-ergocryptine, ergotoxine, ergocornine, ergocristine, ergocryptine, α-ergocryptine, β-ergocryptine, ergosine, ergostane, bromocriptine, or methysergide.

Additionally, the present compounds may be used in conjunction with a beta-adrenergic antagonist such as timolol, propanolol, atenolol, or nadolol, and the like; a MAO inhibitor, for example phenelzine; a calcium channel blocker, for example flunarizine, nimodipine, lomerizine, verapamil, nifedipine, prochlorperazine or gabapentin; neuroleptics such as olanzapine and quetiapine; an anticonvulsant such as topiramate, zonisamide, tonabersat, carabersat or divalproex sodium; an angiotensin I1 antagonist, for example losartan and candesartan cilexetil; an angiotensin converting enzyme inhibitor such as lisinopril; or botulinum toxin type A.

The present compounds may be used in conjunction with a potentiator such as caffeine, an H2-antagonist, simethicone, aluminum or magnesium hydroxide; a decongestant such as phenylephrine, phenylpropanolamine, pseudoephedrine, oxymetazoline, epinephrine, naphazoline, xylometazoline, propylhexedrine, or levo-desoxy-ephedrine; an antitussive such as codeine, hydrocodone, caramiphen, carbetapentane, or dextromethorphan; a diuretic; a prokinetic agent such as metoclopramide or domperidone, and a sedating or non-sedating antihistamine.

In a particularly preferred embodiment the present compounds are used in conjunction with an anti-migraine agent, such as: an ergotamine; a 5-HT₁ agonist, especially a 5-HT_{1B/1D} agonist, in particular, sumatriptan, naratriptan, zolmitriptan, eletriptan, almotriptan, frovatriptan, donitriptan and rizatriptan; and a cyclooxygenase inhibitor, such as a selective cyclooxygenase-2 inhibitor, in particular, rofecoxib, etoricoxib, celecoxib, meloxicam, valdecoxib or paracoxib.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the other active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), and via the same or different routes of administration.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable nonirritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. Similarly, transdermal patches may also be used for topical administration.

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require antagonism of CGRP receptor activity an appropriate dosage level will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, or may be administered once or twice per day.

When treating, preventing, controlling, ameliorating, or reducing the risk of headache, migraine, cluster headache, or other diseases for which compounds of the present invention are indicated, generally satisfactory results are obtained when the compounds of the present invention are administered at a daily dosage of from about 0.1 milligram to about 100 milligram per kilogram of animal body weight, given as a single daily dose or in divided doses two to six times a day, or in sustained release form. For most large mammals, the total daily dosage is from about 1.0 milligrams to about 1000 milligrams, or from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein.

The compounds of the present invention can be prepared readily according to the following Schemes and specific examples, or modifications thereof, using readily available starting materials, reagents and conventional synthesis procedures. In these reactions, it is also possible to make use of variants which are themselves known to those of ordinary skill in this art but are not mentioned in greater detail. The general procedures for making the compounds claimed in this invention can be readily understood and appreciated by one skilled in the art from viewing the following Schemes.

Scheme 1 is illustrative of a general route to compounds of the present invention. The carboxylic acid starting material 1 is initially converted to the corresponding methyl ester using standard procedures. Other esters may be used as protected versions of acid 1, such as ethyl ester or tert-butyl ester, and methods for their synthesis are well known in the art. Bromination of 2 using bromine, or another brominating agent such as N-bromosuccinimide, affords the bromopyridine derivative 3, which is a convenient intermediate for the introduction of a variety of R² groups. In Scheme 1, a copper-promoted coupling reaction is used to give intermediate 4, but it may be readily understood by one skilled in the art that a variety of different reagents and conditions may be applied to 3 to afford a diverse set of possible products. The choice of conditions would depend upon the precise nature of R² and may include reactions promoted by copper, nickel, or palladium catalysts using a variety of bases and solvents. Saponification of ester 4 provides acid 5, which may be coupled to amine R¹NH₂ using EDC-HOBT conditions to give structure 6, a compound of the present invention.

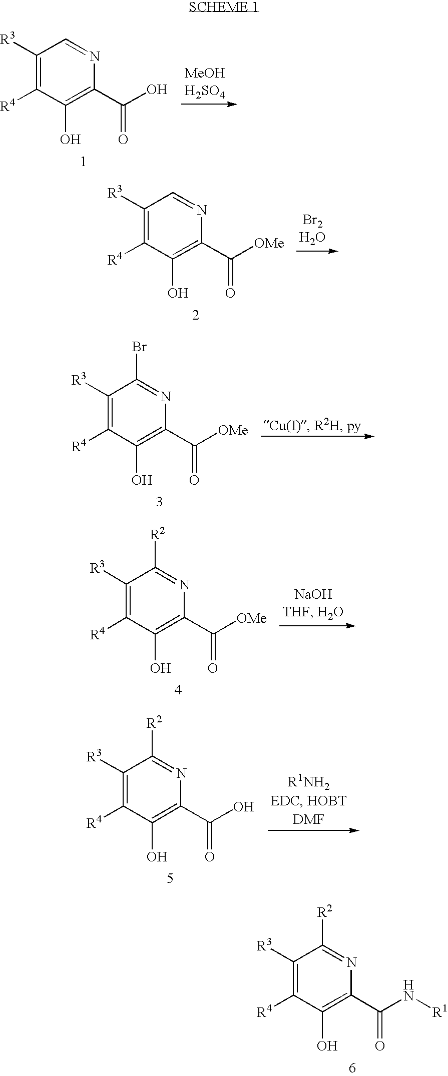

Alternative methods of coupling acid 5 with amines include, but are not limited to, the use of other coupling reagents, such as PyBOP, activation of 5 with phosgene, or conversion of 5 to the corresponding acid chloride or pentafluorophenyl ester. Another method for synthesizing amides like 6 is the direct condensation of amine R¹NH₂ with the ester intermediate 4, for example by heating the reactants in toluene.

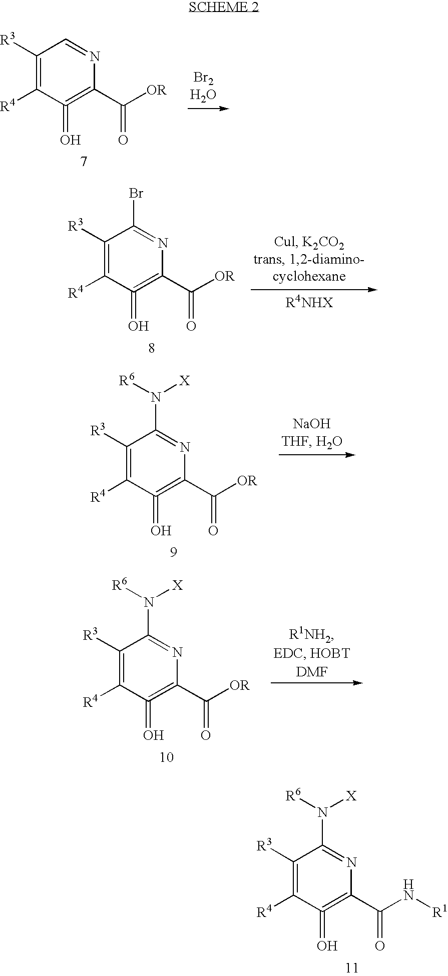

In Scheme 2, bromination of pyridine 7 affords 8, which may be coupled with 5 amides [X═C(O)R⁷], sulfonamides [X═S(O)₂R⁷], or sulfonyl ureas [X═S(O)₂N(R⁶)(R⁷)] in a copper-promoted reaction as shown, to give a variety of products represented by structure 9. Saponification of the ester, followed by coupling to the amine R¹NH₂ provides compounds of the present invention.

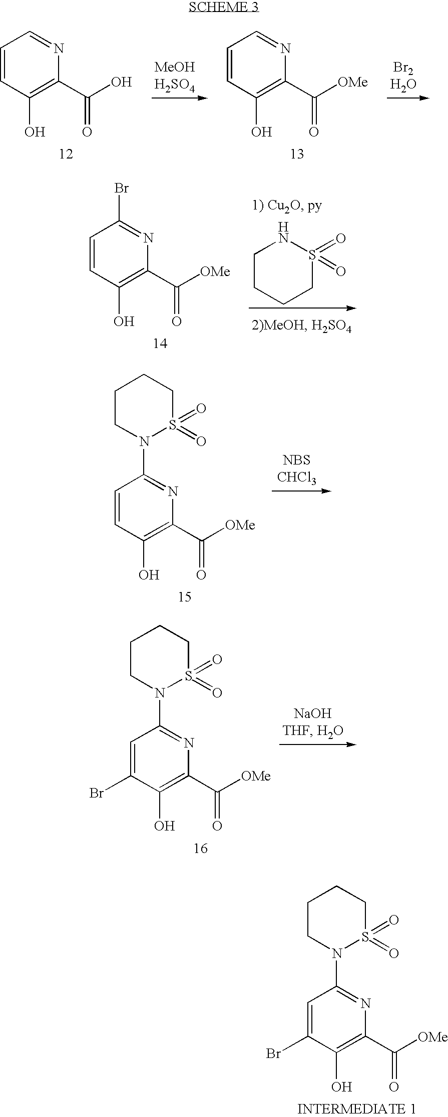

Scheme 3 details the synthesis of Intermediate 1, which is useful for the construction of the compounds of the present invention. Esterification of the commercially available acid 12, followed by bromination, gives bromopyridine 14. Reaction of 14 with δ-sultam and copper (I) oxide in pyridine, followed by re-protection of the carboxylic acid moiety, affords the ester 15. Use of N-bromosuccinimide in chloroform may be used to provide the bromophenol 16, and this may be saponified to give the key Intermediate 1.

The elaboration of Intermediate 1 to give compounds of the present invention is shown in Scheme 4. In the example shown, coupling of the acid with 3,5-difluorobenzylamine using EDC and HOBT produces the compound described in Example 1, below. The Example 1 compound may be subjected to palladium-catalyzed carbonylation in methanol to give another compound of interest, the compound described in Example 11.

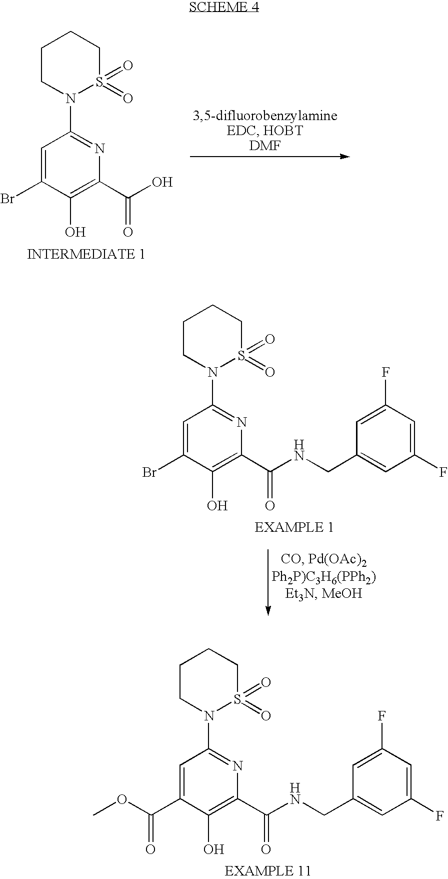

Straightforward modifications of the methodology shown in Scheme 4 can be used to provide the other Examples described herein. For example, use of a different amine or carboxylic acid in the coupling reaction can provide a variety of final amide products. Alternative coupling methodology may also be used to provide such amides. Additionally, the carbonylation conditions used to provide the Example 11 compound may be applied to a variety of bromides to provide other compounds of the present invention.

In some cases the final product may be even further modified, for example, by manipulation of substituents. These manipulations may include, but are not limited to, reduction, oxidation, alkylation, acylation, and hydrolysis reactions which are commonly known to those skilled in the art.

In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Intermediate 1

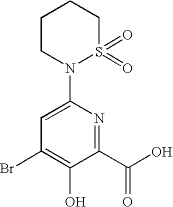

### 4-Bromo-6-(1,1-dioxido-1,2-thiazinan-2-yl)-3-hydroxypyridine-2-carboxylic acid

Step A. Methyl 3-hydroxypyridine-2-carboxylate

A stirred mixture of 3-hydroxypyridine-2-carboxylic acid (11.8 g, 84.8 mmol), and conc. H₂SO₄ (16.6 g, 170 mol) in MeOH (75 mL) was heated to reflux for 18 h, then allowed to cool to ambient temperature. Most of the solvent was removed in vacuo and the residual mixture was partitioned between CH₂Cl₂ (500 mL) and saturated NaHCO₃ (500 mL). The aqueous layer was extracted further with CH₂Cl₂ (3×300 mL) and the combined organic extracts were dried (Na₂SO₄), filtered, and concentrated in vacuo to afford the title compound as a white solid. MS: m/z=154 (M+1).

Step B. Methyl 6-bromo-3-hydroxypyridine-2-carboxylate

To a stirred solution of methyl 3-hydroxypyridine-2-carboxylate (9.03 g, 59.0 mmol) in H₂O (400 mL), at ambient temperature, was added dropwise bromine (12.8 g, 4.10 mL, 80.0 mmol). The mixture was stirred for 3 h, during which time a fine white precipitate formed. The aqueous mixture was extracted with CH₂Cl₂ (2×500 mL) and the combined organic extracts were dried (Na₂SO₄), filtered, and concentrated in vacuo to afford the title compound as a white solid of sufficient purity for use in the next step. MS: m/z=232 (M+1).

### Step C. Methyl 6-(1,1-dioxido-1,2-thiazinan-2-yl)-3-hydroxypyridine-2-carboxylate

A stirred mixture of methyl 6-bromo-3-hydroxypyridine-2-carboxylate (6.15 g, 26.5 mmol), δ-sultam (described in WO 02/30931-A2, Merck & Co., Inc., 2002) (3.98 g, 29.4 mmol) and copper (I) oxide (5.75 g, 40.2 mmol) in anhydrous pyridine (100 mL) was heated at 130° C., under argon, for 6 h. The mixture was cooled, and the pyridine removed under reduced pressure. The residue was treated with CH₂Cl₂ (400 mL) and EDTA (0.35 M in H₂O, 300 mL, 105 mmol) and air was bubbled into the mixture for 18 h. The mixture was filtered through a pad of celite and the aqueous layer was saturated with solid NaCl and extracted with CH₂Cl₂ (4×250 mL). The combined organic extracts were dried (Na₂SO₄), filtered, and concentrated in vacuo to afford a crude oil. The oil was dissolved in MeOH (400 mL) and conc. H₂SO₄ (2 mL) and heated to reflux for 18 h, then allowed to cool to ambient temperature. Most of the solvent was removed in vacuo and the residual mixture was partitioned between CH₂Cl₂ (400 mL) and saturated NaHCO₃ (400 mL). The aqueous layer was extracted further with CH₂Cl₂ (2×250 mL) and the combined organic extracts were dried (Na₂SO₄), filtered, and concentrated in vacuo to afford a crude sample of the product. This crude product was partially purified by silica gel chromatography, eluting with a gradient of CH₂Cl₂:MeOH—100:0 to 95:5 to give a yellow solid that was crystallized from MeOH to afford the title compound as a pale yellow solid of sufficient purity for use in the next step. MS: m/z=287 (M+1).

### Step D. Methyl 4bromo-6-(1,1-dioxido-1,2-thiazinan-2-yl)-3-hydroxypyridine-2-carboxylate

A stirred mixture of methyl 6-(1,1-dioxido-1,2-thiazinan-2-yl)-3-hydroxypyridine-2-carboxylate (2.50 g, 8.73 mmol), and N-bromosuccinimide (2.50 g, 14.1 mmol) in anhydrous CHCl₃ (50 mL) was heated at reflux, under argon, for 3 h. The mixture was cooled and partitioned between EtOAc (200 mL) and saturated NaHCO₃ (100 mL). The aqueous layer was extracted further with EtOAc (200 mL) and the combined organic extracts were dried (Na₂SO₄), filtered, and concentrated in vacuo to afford a crude solid. This solid was triturated with MeOH to afford the title compound as a tan solid. MS: m/z=365 (M+1).

### Step E. 4-Bromo-6-(1,1-dioxido-1,2-thiazinan-2-yl)-3-hydroxypyridine-2-carboxylic acid

A solution of methyl 4-bromo-6-(1,1-dioxido-1,2-thiazinan-2-yl)-3-hydroxypyridine-2-carboxylate (1.50 g, 4.11 mmol), and sodium hydroxide (361 mg, 9.04 mmol) in THF (35 mL) and H₂O (10 mL) was stirred at ambient temperature for 18 h. The mixture was extracted with CHCl₃ (30 mL) and the organic layer was discarded. The aqueous layer was adjusted to pH=3 with aqueous HCl and extracted with CHCl₃ (2×50 mL) and the combined organic extracts were dried (Na₂SO₄), filtered, and concentrated in vacuo to afford the title compound as a yellow solid. MS: m/z=351 (M+1).

### EXAMPLE 1

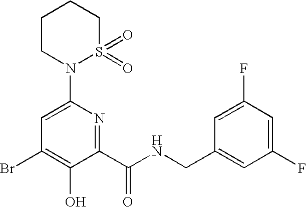

### 4-Bromo-N-(3,5-difluorobenzyl)-6-(1,1-dioxido-1,2-thiazinan-2-yl)-3-hydroxypyridine-2-carboxamide

### Step A. 4-Bromo-N-(3,5-difluorobenzyl)-6-(1,1-dioxido-1,2-thiazinan-2-yl)-3-hydroxypyridine-2-carboxamide

A mixture of 4-bromo-6-(1,1-dioxido-1,2-thiazinan-2-yl)-3-hydroxypyridine-2-carboxylic acid (Intermediate 1) (144 mg, 0.41 mmol), EDC (173 mg, 0.90 mmol), and HOBT (75 mg, 0.49 mmol) was stirred in DMP (2 mL) at ambient temperature for 1 min, then 3,5-difluorobenzylamine (177 mg, 1.23 mmol) was added. The mixture was stirred for 2 h, then partitioned between CH₂Cl₂ (50 mL) and saturated NaHCO₃ (25 mL). The aqueous layer was extracted further with CH₂Cl₂ (2×50 mL) and the combined organic extracts were dried (Na₂SO₄), filtered, and concentrated in vacuo. The crude product was purified by HPLC using a reversed phase C18 column and eluting with a gradient of H₂O:CH₃CN:CF₃CO₂H—90:10:0.1 to 5:95:0.1. Lyophilization provided the title compound as a pale solid. MS: m/z=476 (M+1). HRMS: m/z=476.0099; calculated m/z=476.0086 for C₁₇H₁₇BrF₂N₃O₄S.

### EXAMPLES 2-7

Essentially following the procedures outlined for Example 1, the compounds listed in Table 1 were prepared. The requisite amines were commercially available, described in the literature, or readily synthesized by one skilled in the art of organic synthesis. In some cases, straightforward protecting group strategies were applied.

TABLE 1

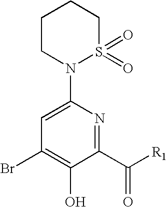

| Example | R₁ | MS (M + 1) |
|---|---|---|
| 2 | 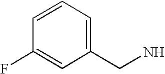 | 458 |
| 3 | 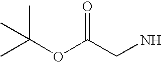 | 464 |
| 4 | 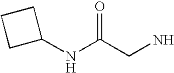 | 461 |
| 5 | 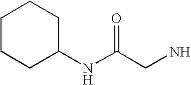 | 489 |
| 6 | 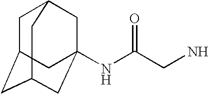 | 541 |
| 7 | 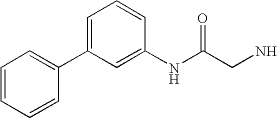 | 559 |

### EXAMPLE 8

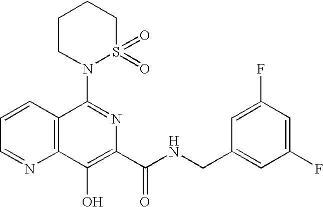

### N-(3,5-Difluorobenzyl)-5-(1,1-dioxido-1,2-thiazinan-2-yl)-8-hydroxy-1,6-naphthyridine-7-carboxamide

### Step A. N-(3,5-Difluorobenzyl)-5-(1,1-dioxido-1,2-thiazinan-2-yl)-8-hydroxy-1,6-naphthyridine-7-carboxamide

Following the procedures outlined for Example 1, but using 5-(1,1-dioxido-1,2-thiazinan-2-yl)-8-hydroxy-1,6-naphthyridine-2-carboxylic acid (described in WO 02130931-A2, Merck & Co., Inc., 2002) in place of Intermediate 1, the title compound was obtained as a pale solid. MS: m/z=449 (M+1). HRMS: m/z=449.1075; calculated m/z=449.1090 for C₂₀H₁₉F₂N₄O₄S.

### EXAMPLES 9-10

Essentially following the procedures outlined for Example 1, the compounds listed in Table 2 were prepared. The requisite amines were commercially available, described in the literature, or readily synthesized by one skilled in the art of organic synthesis.

TABLE 2

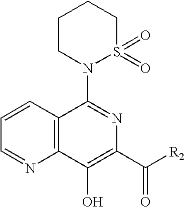

| Example | R₂ | MS (M + 1) |
|---|---|---|
| 9 | 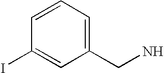 | 539 |
| 10 | 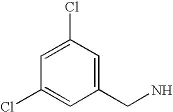 | 481 |

### EXAMPLE 11

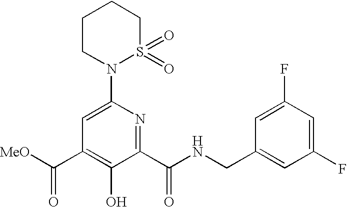

### Methyl 2-{[(3,5-difluorobenzyl)amino]carbonyl}-6-(1,1-dioxido-1,2-thiazinan-2-yl)-3-hydroxyisonicotinate

### Step A. Methyl 2-{[(3,5-difluorobenzyl)amino]carbonyl}-6-(1,1-dioxido-1,2-thiazinan-2-yl)-3-hydroxyisonicotinate

A stirred mixture of 4-bromo-N-(3,5-difluorobenzyl)-6-(1,1-dioxido-1,2-thiazinan-2-yl)-3-hydroxypyridine-2-carboxamide (Example 1) (100 mg, 0.210 mmol), Pd(OAc)₂ (24 mg, 0.105 mmol), 1,3-bis(diphenylphosphino)propane (43 mg, 0.105 mmol) and triethylamine (0.15 mL, 1.08 mmol) in MeOH (7 mL) was heated at a gentle reflux under an atmosphere of CO (ca. 1 atm) for 18 h. The reaction mixture was concentrated to dryness and the residue was resuspended in EtOAc (20 mL) and washed with 10% aqueous citric acid (5 mL), then brine (5 mL) and the organic layer was dried (Na₂SO₄), filtered, and concentrated in vacuo. The crude product was purified by HPLC using a reversed phase C18 column and eluting with a gradient of H₂O:CH₃CN:CF₃CO₂H—90:10:0.1 to 5:95:0.1. Lyophilization provided a solid that was further purified by silica gel chromatography, eluting with a gradient of hexane:EtOAc:HCO₂H—80:20:2 to 50:50:2 to give the title compound as a white solid. MS: m/z=456 (M+1). HRMS: m/z=456.1048; calculated m/z=456.1036 for C₁₉H₂₀P₂N₃O₆S.

### EXAMPLE 12

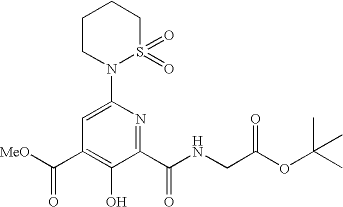

### Methyl 2-({[(tert-butoxycarbonyl)methyl]amino}carbonyl)-6-(1,1-dioxido-1,2-thiazinan-2-yl)-3-hydroxyisonicotinate

### Step A. Methyl 2-({[(tert-butoxycarbonyl)methyl]amino}carbonyl)-6-(1,1-dioxido-1,2-thiazinan-2-yl)-3-hydroxyisonicotinate

Following the procedures outlined for Example 11, but using Example 3 in place of Example 1, the title compound was obtained. MS: m/z=444 (M+1). HRMS: m/z=444.1440; calculated m/z=444.1435 for C₁₈H₂₆N₃O₈S.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. Therefore, the invention is defined by the claims which follow and not limited by the examples.

What is claimed is:

1. A compounds of the Formula: 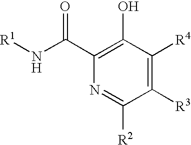
wherein:
R¹ is selected from:
a) hydrogen,
b) aryl, heterocycle, C₃-C₁₀ cycloalkyl, C₂-C₆ alkenyl, C₂-C₆ alkynyl, and
c) C₁-C₆ alkyl, unsubstituted or substituted with 1 to 5 substituents selected from:
1) aryl, unsubstituted or substituted with 1 to 5 substituents selected from:
i) C₁-C₆ alkyl, unsubstituted or substituted with 1-3 fluoro,
ii) C₃-C₆ cycloalkyl,
iii) C₂-C₆ alkynyl,
iv) OR¹⁰,
v) aryl,
vi) heterocycle,
vii) CN, and
viii) halo;
2) heterocycle, unsubstituted or substituted with 1 to 5 substituents selected from:
i) C₁-C₆ alkyl, unsubstituted or substituted with 1-3 fluoro,
ii) —OR¹⁰,
iii) aryl, and
iv) halo;
3) C₃-C₁₀ cycloalkyl,
4) C₂-C₆ alkenyl,
5) C₂-C₆ alkynyl,
6) —OR¹⁰,
7) —S(O)ₘR¹¹,
8) —NR⁶—C(O)R⁷,
9) —C(O)—N(R⁶)(R⁷),
10) —CN,
11) —NR⁶—C(O)N(R⁶)(R⁷),
12) —C(O)—OR¹⁰,
13) halo, and
14) —N(R⁶)(R⁷);
R² is selected from:
a) —NR⁶—C(O)R⁷,
b) —NR⁶—S(O)₂R⁷, and
d) c) —NR⁶—S(O)²—N(R⁶)(R⁷);
R³ and R⁴ are independently selected from:
hydrogen, aryl, heterocycle, halo, C₁-C₆ alkyl, C₃-C₁₀ cycloalkyl, C₂-C₆ alkenyl, C₂-C₆ alkynyl, C₁-C₄ haloalkyl, R¹⁰O—, R¹¹S(O)ₘ—, R⁶C(O)—NR⁷—, CN, (R⁶)(R⁷)N—C(O)—(NR⁶)—, (R⁶)(R⁷)—N—C(O)—, R¹⁰C(O)—, R¹⁰OC(O)—, and N(R⁶)(R⁷); or
wherein R³ and R⁴ are optionally joined to form a saturated or unsaturated ring, containing 0-3 heteroatoms, wherein said ring is phenyl, pyridyl, pyrimidinyl, pyrazinyl, thiophenyl, furanyl, imidazolyl, thiazolyl, oxazolyl, and triazolyl, as well as partially saturated analogues thereof, said ring optionally substituted with one or more of:
aryl, heterocycle, C₁-C₆ alkyl, C₃-C₁₀ cycloalkyl, C₂-C₆ alkynyl, R¹⁰O—, R¹¹S(O)ₘ—, R⁶C(O)NR⁷—, R⁶S(O)2NR⁷—, (R⁶)(R⁷)N—C(O)—, CN, R¹⁰OC(O)—, F, and N(R⁶)(R⁷);
R⁶ and R⁷ are independently selected from hydrogen, C₁-C₆ alkyl, C₃-C₁₀ cycloalkyl, heterocycle, aryl, unsubstituted or substituted with one or more of:
a) C₁-C₄ alkyl,
b) C₁-C₄ alkoxy,
c) aryl or heterocycle,
d) halo,
e) —OR¹⁰, and
f) —N(R¹⁰)₂;
wherein R⁶ and R⁷ may be joined to form a ring;
R¹⁰ is independently selected from hydrogen, C₁-C₆ alkyl, -CF₃, C₃-C₁₀ cycloalkyl, benzyl, and aryl;
R¹¹ is independently selected from C₁-C₆ alkyl, and aryl;
m is 0, 1, or 2;
and pharmaceutically acceptable salts and individual diastereomers thereof.

2. The compound according to claim 1, wherein R¹ is —CH₂-aryl, unsubstituted or substituted with 1-3 substituents selected from: fluoro, chloro, bromo, iodo and methyl.

3. The compound according to claim 1, wherein R¹ is benzyl, substituted with 1-3 fluoro.

4. The compound according to claim 1, wherein R¹ is —CH₂C(O)OR¹⁰.

5. The compound according to claim 1, wherein R¹ is —CH₂C(O)OC(CH₃)₃.

6. The compound according to claim 1, wherein R¹ is —CH₂C(O)NHR⁶.

7. The compound according to claim 1, wherein R¹ is —CH₂C(O)NH(C₄-C₁₀ cycloalkyl).

8. The compound according to claim 1, wherein R¹ is —CH₂C(O)NH-aryl.

9. The compound according to claim 1, wherein R² is —NR⁶-S(O)₂R⁷.

10. The compound according to claim 1, wherein R³ is hydrogen.

11. The compound according to claim 1, wherein R³ and R⁴ are joined to form a ring selected from: phenyl, pyridyl, pyrimidinyl and pyrazinyl.

12. The compound according to claim 1, wherein R³ and R⁴ are joined to form a pyridyl ring.

13. The compound according to claim 1, wherein R⁴ is bromo.

14. The compound according to claim 1, wherein R⁴ is —C(O)OR¹⁰.

15. A compound selected from: 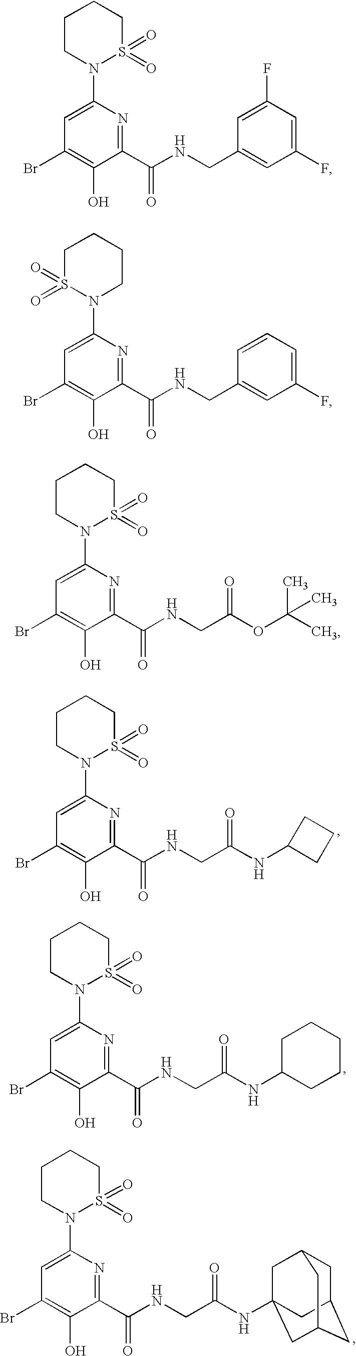 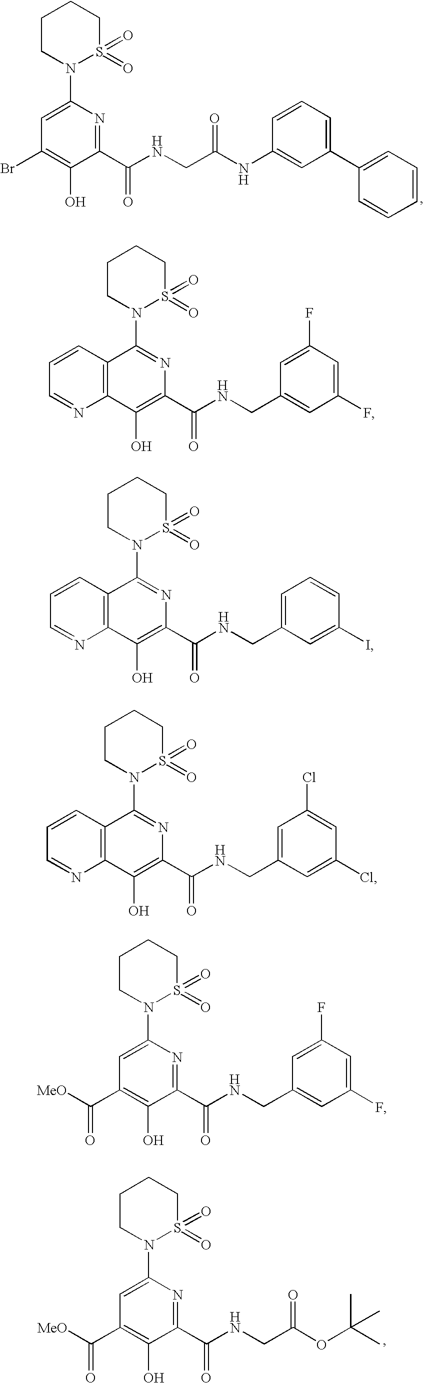
and pharmaceutically acceptable salts and individual diastereomers thereof.

16. A pharmaceutical composition which comprises an inert carrier and the compound of claim 1.

17. A method for antagonism of CGRP receptor activity in a mammal which comprises the administration of an effective amount of the compound of claim 1.

18. A method for treating, controlling, ameliorating or reducing the risk of headache, migraine or cluster headache in a mammalian patient in need of such which comprises administering to the patient a therapeutically effective amount of the compound of claim 1.

19. A method for modulation of AM receptor activity in a mammal which comprises the administration of an effective amount of the compound of claim 1.

20. A method for treating, controlling, ameliorating or reducing the risk of cancer, diabetic retinopathy, vascular disorders, heart failure, septic shock, hypertension, renal failure and diabetes in a mammalian patient in need of such which comprises administering to the patient a therapeutically effective amount of the compound of claim 1.

21. A method of treating or preventing migraine headaches, cluster headaches, and headaches, said method comprising the co-administration, to a person in need of such treatment, of:
a therapeutically effective amount of the compound of claim 1 or a pharmaceutically acceptable salt thereof; and
a therapeutically effective amount of a second agent selected from serotonin agonists, analgesics, anti-inflamatory agents, anti-hypertensives and anticonvulsants.

22. The method of claim 21, wherein said second agent is selected from a 5HT_{1B/1D} agonist, a 5HT_{1D} agonist, a 5HT_{1F} agonist, ergotamine, dihydroergotamine, aspirin, acetaminophen, a glucocorticoid, a non-steroidal anti-inflammatory agent.

23. The method of claim 21, wherein said second agent is selected from angiotensin II antagonists, angiotensin I antagonists, angiotensin converting enzyme inhibitors, and renin inhibitors.

24. A method of treating or preventing migraine headaches, cluster headaches, and headaches, said method comprising the co-administration, to a person in need of such treatment, of:
a therapeutically effective amount of the compound of claim 1 or a pharmaceutically acceptable salt thereof; and
a therapeutically effective amount of a second agent selected from anti-anxiety agents, neuroleptics, beta-blockers, calcium channel blockers, anti-depressants, selective serotonin reuptake inhibitors, NE reuptake inhibitors, botulinum toxins A or B, vanilloid receptor antagonists, adenosine 1 antagonists, NR2B antagonists, substance P antagonists, granzyme B inhibitors, endothelin antagonists, norepinephrin precursors, nitric oxide synthase inhibitors, neuroleptics, bradykinin antagonists, gap junction inhibitors, AMPA/KA antagonists, sigma receptor agonists, chloride channel enhancers, monoamine oxidase inhibitors, opioid agonists, and leukotriene receptor antagonists, anti-emetics, prokinetics, and histamine H1 antagonists.
